(19) United States
(12) Patent Application Publication
(10) Pub. No.: US 2018/0305283 A1
(43) Pub. Date: Oct. 25, 2018

(54) Process for Preparing 2,6-Substituted Phenols

(71) Applicant: PPP Chemistry B.V., Apeldoom (NL)

(72) Inventor: Mathias Reto Guggisberg, Lent (NL)

(21) Appl. No.: 15/770,923

(22) PCT Filed: Nov. 10, 2016
(86) PCT No.: PCT/EP2016/077237

§ 371 Date: Apr. 25, 2018

(30) Foreign Application Priority Data
Nov. 10, 2015 (NL) 2015765

(51) Int. Cl. C07C 37/18; C07C 39/15; B01J 31/24; B01J 31/12

(52) U.S. Cl. CPC C07C 37/18; C07C 39/15; B01J 31/2404; C08G 65/485; C07C 2531/24; B01J 2231/4227; B01J 2531/824; B01J 31/124

(57) ABSTRACT

The present invention relates to a process for preparing 2,6-substituted phenols, and in particular to a process for preparing 2,6-diphenylphenol. This process is a doubling coupling of a boronic acid and a 2,6-dihalogenphenol in a Suzuki-Miyaura reaction on sterically hindered ortho positions. In a preferred embodiment, this process takes place in a continuous flow system. The present invention further relates to the composition obtained by this process, and to the use of this composition for preparing poly(2,6-diphenylphenylene oxide), for the manufacture of dyes, drugs, plastics, insulating materials and/or insecticides, and for use in medical applications and material research.

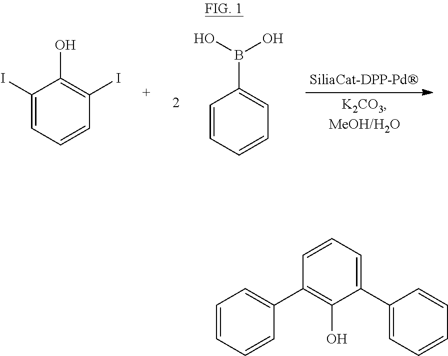 [SMILES: Oc1c(I)cccc1I ; OB(O)c1ccccc1 ; Oc1c(-c2ccccc2)cccc1-c1ccccc1]

The present invention relates to a process for preparing 2,6-substituted phenols, in particular 2,6-diphenylphenol, a composition obtained by the process and use of the composition for preparing poly(2,6-diphenylphenylene oxide), use of the composition in the manufacture of dyes, drugs, plastics, insulating materials, and insecticides, and use of the composition in medical applications and material research.

2,6-Diphenylphenol is a monomer important in the manufacture of dyes, drugs, plastics, insulating materials, insecticides and the like. 2,6-Diphenylphenol is also used in the preparation of TENAX®, a porous polymer used as a column packing material for trapping volatiles from air and liquids.

2,6-diphenylphenol is currently prepared by a process comprising the auto-condensation of cyclohexanone in the presence of an alkaline catalyst to form a mixture of tricyclic ketones that are dehydrogenated to yield 2,6-diphenylphenol. This process, however, suffers from various drawbacks, such as its elaborate procedure, high costs, the production of large amounts of waste material and a relatively low yield. More importantly, this process is dangerous because cancerogenous solvents are used and there is a high risk of explosions.

In the past two decades, several attempts by the present inventors for improving the process were undertaken, thereby focusing on costs, efficiency, higher yield and greener chemistry. A traditional organic approach via acrolein condensation with dibenzylketone did indeed cut the expenses but did not improve the yield and reduce waste materials sufficiently. Rhodium catalyzed coupling of bromobenzene and phenol led to a higher yield of about 70-80% and less waste, but to make it commercially attractive the very expensive rhodium needs to be suitable for re-use, which was not achieved.

It is an object of the present invention to provide a process for preparing 2,6-substituted phenols, in particular 2,6-diphenylphenol, which process does not have the above-mentioned drawbacks. More specifically, it is an object of the present invention to provide a more efficient process for preparing 2,6-substituted phenols, in particular 2,6-diphenylphenol, which process is quicker, safer, cost efficient and greener than the currently used process.

This object is achieved by double coupling of a boronic acid and a 2,6-dihalogenphenol in a Suzuki-Miyaura reaction on a sterically hindered position. In the research that led to the present invention green solvents, such as water and methanol, and a catalyst, in particular a palladium catalyst, were used to couple phenyl boronic acid and 2,6-diiodophenol in a batch Suzuki-Miyaura reaction. This reaction resulted in a yield of about 80% 2,6-diphenylphenol. The catalyst still works after completion of the reaction and can thus be used again. Further, if the reaction is performed in a continuous flow chemistry setting, a conversion rate of 100% is achieved.

The present invention thus relates to a process for preparing a compound of formula (I),

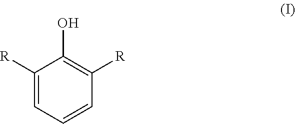

wherein R represents phenyl, substituted aryl, alkyl, or substituted alkyl. The R on position 2 may be the same as, or different than, the R on position 6,
comprising, reacting a compound of formula (II)

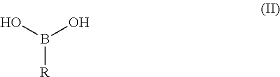

wherein R represents phenyl, substituted aryl, alkyl, or substituted alkyl,
with a compound of formula (III)

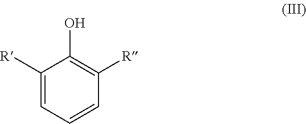

wherein R′ and/or R″ represents a halogen,
in the presence of a catalyst.

This reaction is a double coupling of a boronic acid and a 2,6-dihalogenphenol in a Suzuki-Miyaura reaction on a sterically hindered position, in particular on the sterically hindered ortho positions.

The compound of formula (II) can be any boronic acid. A preferred compound of formula (II) is phenyl boronic acid.

The compound of formula (III) can be any 2,6-dihalogenphenol, i.e. any phenol having a halogen at positions 2 and 6. The halogen can be any halogen. The halogen at position 2, indicated as R′, can be the same or a different halogen as the halogen at position 6, indicated as R″. Preferably, the halogen is iodine (I), bromide (Br) or chloride (Cl). A preferred compound of formula (III) is 2,6-diiodophenol.

In one embodiment, the compound of formula (I) is 2,6-diphenylphenol, the compound of formula (II) is phenyl boronic acid and the compound of formula (III) is 2,6-diiodophenol.

The catalyst is preferably a palladium catalyst. A palladium catalyst surprisingly catalyzes the coupling of a boronic acid and a 2,6-dihalogenphenol at the sterically hindered ortho positions in relation to the hydroxyl group, in particular at the sterically hindered ortho positions of the 2,6-dihalogenphenol. More preferably, the catalyst is SiliaCat-DPP-Pd.

The catalyst may be a homogenous or a heterogenous catalyst. A heterogenous catalyst does not dissolve in a liquid and is therefore preferred in the process of the present invention. A homogenous catalyst dissolves in a liquid and is therefore not as easy or efficient to extract from the reaction mixture.

The concentration at which the catalyst is to be used can be determined by a person skilled in the art. A suitable concentration ratio, in part catalyst to part product (2,6-dihalogenphenol), is for instance between 0.5:100 and 2:100, and preferably 1:100. More than 2 parts catalyst and 100 parts product is also possible although this may economically be too expensive.

A major advantage of a palladium catalyst is that it can be reused, making it also suitable for use in a continuous flow system. A palladium catalyst can remain in a flow chemistry device and does not need to be separated after the reaction. This can be achieved by using the catalyst on a solid support. In batch mode, the catalyst can be removed by filtration.

As is known to a person skilled in the art, a Suzuki-Miyaura reaction needs to be performed in the presence of a base. Any suitable base can be used, such as for instance K₂CO₃, NaOH and triethylamine.

The process can be performed in any suitable manner, such as for instance in batches or in a continuous flow system. It is preferred to perform the method in a continuous flow system. When a continuous flow system, such as a continuous flow reactor, is used, the yield of 2,6-substituted phenol, in particular 2,6-diphenylphenol, can be increased to at least 85%, 90%, 95%, 96%, 97%, 98%, 99%, 99.5%, 99.9%, or even 100% by weight.

Using continuous flow chemistry has the advantage that the reaction time is much shorter and it provides further improved safety due to the small reaction volume. Furthermore, the reaction conditions can be set very accurately due to the fast mass and temperature transfer. Due to the small footprint of continuous flow chemistry devices, savings can be achieved on upfront installation costs. Scaling-up is also relatively easy to achieve due to the identical reaction conditions. The parameters of the continuous flow device can be set very accurately, so that the exact reaction conditions are known and can be easily controlled. A process developed on a milligram scale can quickly be scaled-up to kilograms or tons by selecting a suitable continuous flow device. Scaling-up does not jeopardize the improved safety as the continuous flow reactor is still small in size relative to the large tanks used when scaling-up batch processes.

The reaction time with the process of the invention is less than 6 hours. In a batch process, reaction times of between 10 minutes to 6 hours have shown to result in acceptable yields. For instance, a reaction time of about 10 minutes resulted in a yield of 80% by weight, and a reaction time of about 30 minutes resulted in a yield of 85% by weight. In both cases the temperature of the batch reaction was about 55° C.

In a continuous flow system, reaction times are even reduced to 1.2 to 4.8 minutes. For instance, a reaction time of 4.8 minutes resulted in a yield of 100% by weight. In one embodiment, a yield of at least 75% by weight, preferably more than 96% by weight, is obtained by performing the process of the invention at a temperature of between 20° C. to higher than 100° C., preferably 25-100° C., more preferably 50-100° C., and even more preferably about 100° C., and a reaction time of between 1.2 and 4.8 minutes, and preferably a reaction time of about 4.8 minutes. In another embodiment, a yield of at least 99% by weight is obtained by performing the process of the invention at a temperature of about 100° C., and a reaction time of about 4.8 minutes.

The reaction time with the process of the invention is therefore suitably between 1 minute and 6 hours. This low reaction time is a major improvement compared to the reaction times described in the prior art.

The ‘yield’ in the context of the invention is the percentage by weight of the desired product in the resulting composition. A yield of at least 85% thus means that 85% of the composition resulting from the process of the invention consists of 2,6-substituted phenol. In other words, at least 85% of the starting material, in the present case a boronic acid and a 2,6-dihalogenphenol, is converted into the desired product, which in the present case is a 2,6-substituted phenol.

The temperature at which the process can be performed can be varied as required as is known to a person skilled in the art. Suitable temperatures for performing the process of the invention in a continuous flow system wherein a yield of at least 75% by weight is desired are 20° C. to higher than 100° C., preferably 25-100° C., and more preferably 50-100° C.

The process of the present invention has clear advantages over the existing processes for preparing 2,6-substituted phenols, in particular 2,6-diphenylphenol. Firstly, a higher yield is achieved. Secondly, the reaction time is much shorter. Thirdly, it is environmentally friendly and safer as green solvents and unhazardous reagents are used. Fourthly, it is more costs effective as cheaper solvents, reagents and catalysts are used and can be recycled as a consequence of the given mild reaction conditions.

The process according to the invention results in a composition comprising at least 75% 2,6-substituted phenol, in particular 2,6-diphenylphenol. When the process is performed in a continuous flow system, the resulting composition comprises at least 85%, 90%, 95%, 96%, 97%, 98%, 99%, 99.5%, 99.9%, or even 100% 2,6-substituted phenol, in particular 2,6-diphenylphenol. The present invention therefore also relates to a composition obtained by the process according to the invention. This composition is also referred to herein as “the composition of the present invention” or “the composition of the invention”. The composition of the present invention thus comprises a high amount of 2,6-substituted phenol, in particular 2,6-diphenylphenol.

Its high yield makes the composition obtained by the process of the invention particularly suitable as precursor for the preparation of TENAX®. TENAX® is a porous polymer based on 2,6-diphenylphenylene oxide, that is widely used as a column packing material for trapping volatiles from air (VOC) or liquids. TENAX® is particularly useful for the analysis of high boiling compounds such as alcohols, polyethylene glycols, diols, phenols, monoamines and diamines, ethanolamines, amides, aldehydes, ketones and chlorinated aromatics.

For the preparation of TENAX®, also referred to as poly(2,6-diphenylphenylene oxide), a purity of at least 99.9% is required. It is much easier and more efficient to purify the 2,6-substituted phenol, in particular 2,6-diphenylphenol, from the composition of the present invention, as it has a high amount of 2,6-substituted phenol, in particular 2,6-diphenylphenol, and a low amount of partly substituted phenols. The composition of the present invention is therefore more suitable for the preparation of TENAX® than 2,6-diphenylphenol prepared by other processes.

The present invention thus also relates to the use of the composition of the present invention for preparing poly(2,6-diphenylphenylene oxide).

The present invention also relates to the use of the composition of the present invention for the manufacture of dyes, drugs, plastics, insulating materials, and/or insecticides.

The present invention also relates to the use of the composition of the present invention as a ligand in medical applications and/or material research.

The invention furthermore relates to the use of a palladium catalyst for preparing a 2,6-substituted phenol, and in particular for preparing 2,6-diphenylphenol. Preferably, the palladium catalyst is SiliaCat-DPP-Pd.

### FIGURES

FIG. 1 is a schematic overview of the Suzuki Miyaura reaction of phenyl boronic acid and 2,6-diiodophenol.

### EXAMPLES

### Example 1. Preparation of 2,6-Diphenylphenol

### Provision of Phenyl Boronic Acid

Phenyl boronic acid is available on the market through several suppliers. Alternatively, phenyl boronic acid may be prepared by reacting bromobenzene and n-BuLi in the presence of B(OMe)₃, followed by hydrolysis with sulfuric acid. The reaction can be performed as a batch reaction, but may also be performed in a continuous flow system.

### Preparation of 2,6-Diiodophenol

2,6-Diiodophenol was prepared by reacting 1 eq phenol and 1.5 eq iodine (I₂) and 2 eq 30% hydrogen peroxide (H₂O₂) for 24 hours at room temperature, in accordance with the protocol described in Rafael D. C. Gallo, Karimi S. Gebara, Rozanna M. Muzzi and Cristiano Raminelli, J. Braz. Chem. Soc., Vol. 21, No. 4, 770-774, 2010. The reaction was performed as a batch reaction. The reaction may also be performed in a continuous flow system.

### Preparation of 2,6-diphenylphenol

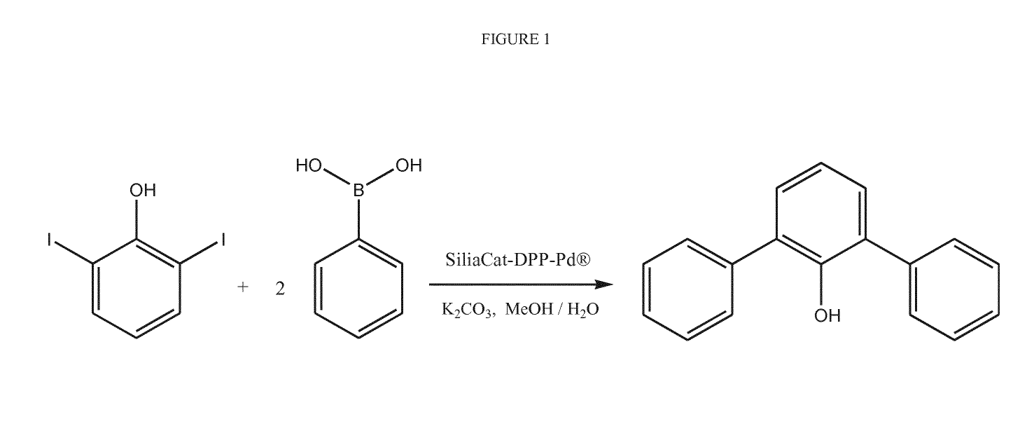

Phenyl boronic acid and 2,6-diiodophenol were used in a Suzuki-Miyaura reaction as shown in FIG. 1.

A mixture of 346 mg (1 mmol) 2,6-diiodophenol, 305 mg (2.5 mmol) phenyl boronic acid, 414 mg potassium carbonate (3 mmol) and 80 mg (1 mol %) SiliaCat-DPP-Pd was suspended in 25 ml of a mixture of methanol/water=8/2. This mixture was stirred at 55° C. for 1 hour.

The reaction was frequently followed on TLC (eluens: hexane/dichloromethane=8/2). After 10 minutes 80% conversion had taken place. When the reaction was completed, the catalyst was filtered off and water was added to the filtrate.

The solution was extracted with ethylacetate, dried on magnesium sulphate and concentrated in vacuum.

The crude mixture was purified on a silicagel column using hexane/dichloromethane=8/2 as eluens to give 191 mg of pure 2,6-diphenylphenol (78% yield).

### Example 2

### Preparation of 2,6-diphenylphenol in a Continuous Flow System

By utilising continuous flow, the aim was to assess if it is possible to perform the Suzuki-Miyaura reaction between 2,6-diiodophenol and phenyl boronic acid in the presence of Si-DPP-Pd catalyst to afford 2,6-diphenylphenol in a shorter time than the analogous batch reaction as described in Example 1. Additional targets included a desire to increase the reaction yield and to reduce the need for separation steps in order to purify the products.

The reaction was assessed utilising a packed-bed flow reactor wherein the effect of flow rate (5-20 μl min−1) and reactor temperature (25 to 100° C.) are assessed on the formation of 2,6-diphenylphenol, using aq. MeOH as the reaction solvent and potassium carbonate as the base.

The following reagents were used: potassium carbonate; 2,6-diiodophenol; Si-DPP-Pd (Silicycle); phenyl boronic acid; 2,6-diphenylphenol; HPLC grade methanol (Fischer Scientific, UK) and de-ionised water.

A Varian GC-MS fitted with a Zebron ZB-5 (30 m (long)×0.25 mm (i.d.)×0.25 μm (film thickness)) capillary column (Phenomenex (UK)) was employed for analysis and quantification of the samples generated using Labtrix Start. Flow reactions were executed using a standard Labtrix Start system fitted with a catalyst set upgrade having PEEK, glass and FFKM wetted parts. The system is capable of investigating flow reactions over a thermal range of −20 to 195° C. at 20 bar and having additional independent pump lines where required. A hand held pressure meter was fitted to inlet A, behind the check valve and ahead of the micro reactor holder, in order to measure the pressure within the reactor during reactions. A glass micro reactor containing a packed-bed (Device 3026) was employed herein.

The internal standards phenyl boronic acid, 2,-6-diiodophenol and 2,6-diphenylphenol were analysed by GC using the following methodology: column=Zebron ZB-5, injection volume=1 μl, split ratio=100:1, injector temperature=200° C., oven temperature 75° C. for 3 min, ramping to 200° C. at 10° C. min⁻¹ and held for 5.50 min (21 min total run time); helium flow rate=1.0 ml min⁻¹. A 2.0 min filament delay was employed and afforded a total run time of 23.5 min.

TABLE 1
Summary of the retention times obtained for the key analytes of
interest as determined via GC-MS analysis.

| Analyte | Retention time (Min) |
|---|---|
| 2,6-Diiodophenol 1 | 15.05 |
| Phenyl boronic acid 2 | 19.37 |
| 2,6-Diphenylphenol 4 | 21.30 |
| Biphenyl 7 | 11.97 |
| Mono-phenylphenol 6 | 18.34 |

The continuous flow reactions were performed using aq. MeOH (8:2 MeOH:H₂O) as reaction solvent and potassium carbonate as the inorganic base. For reasons of limited product solubility at room temperature, a reduced concentration of reactants was employed in the flow reactor (Table 2).

TABLE 2
Composition of reactant solutions.

| Solution # | Composition |
|---|---|
| Standard A | 96.2 mg 2,6-diiodophenol 1 & 82.8 mg K₂CO₃ 5/5 ml |
| | 8:2 (MeOH:H₂O) |
| Standard B | 61 mg phenyl boronic acid 2/5 ml 8:2 (MeOH:H₂O) |

Prior to performing any reactions, the flow reactor was packed with the catalyst (13.3 mg). In order to avoid damage to the reactor any fines (<45 μm) were removed by sieving prior to use. The void volume of the reactor was measured and found to be 24 μl. The reactor was initially flushed with reaction solvent in order to wet the catalyst bed. Subsequently, the reactant solutions were introduced into the reactor at equal flow rates, in order to maintain the reagent stoichiometry utilised in batch (1 eq. 2,6-diiodophenol:2.5 eq. phenyl boronic acid:3 eq. K₂CO₃). Preliminary investigations were performed using a total flow rate of 5 μl min−1 at 25° C. and the reaction product was analysed using offline GC-MS analysis.

Subsequently, the reaction temperature was increased to 50° C., then 75° C. and finally 100° C. At 25° C., two additional analyte peaks were observed at 11.97 min and 18.34 min. Analysis of the mass spectra revealed that these were biphenyl and the mono-phenylphenol intermediate. On increasing the reaction temperature, the mono-phenylphenol intermediate was completely converted to the target 2,6-diphenylphenol. Whilst it was quoted to assess 1 μl min−1, the fact that complete conversion was obtained, this was substituted with 20 μl min−1 to evaluate how short a reaction time was possible.

Table 3 summarises the reaction conversion and selectivities obtained over the conditions assessed. Interestingly, consumption of 2,6-diiodophenol remained high throughout. However, the proportion of mono-phenylphenol intermediate increased with increasing flow rate. This insinuates insufficient residence time within the catalyst bed. A longer reaction time at an elevated temperature is therefore thought to be advantageous. The residence time can be calculated based on the total flow and void volume.

It is assumed that all components have the same relative response factor by GC-MS.

TABLE 3
Reaction conversion at different flow rates and temperatures.

| | Total flow | Temperature | 1 Conv. | Mono-6 | Di-4 |
|---|---|---|---|---|---|
| Run ID | (μl/min⁻¹) | (° C.) | (%) | (%) | (%) |
| 1 | 5 | 25 | 74.1 | 21.5 | 87.5 |
| 2 | 5 | 50 | 88.3 | 0 | 100 |
| 3 | 5 | 75 | Quant. | 0 | 100 |
| 4 | 5 | 100 | Quant. | 0 | 100 |
| 5 | 7.5 | 100 | 98.7 | 0.4 | 99.6 |
| 6 | 7.5 | 75 | 85.2 | 6.2 | 93.8 |
| 7 | 7.5 | 50 | 85.0 | 14.7 | 85.3 |
| 8 | 10 | 50 | 80.1 | 22.8 | 77.2 |
| 9 | 10 | 75 | 78.9 | 9.8 | 90.2 |
| 10 | 10 | 100 | 87.4 | 4.3 | 95.7 |
| 11 | 20 | 100 | 83.9 | 7.7 | 92.3 |
| 12 | 20 | 75 | 81.0 | 11.1 | 88.9 |
| 13 | 20 | 50 | 79.9 | 33.6 | 66.4 |

Using a continuous flow reactor, it was thus observed that 2,6-diiodophenol can be converted to 2,6-diphenylphenol at high conversion rates at temperatures >50° C. When reactions were performed at lower temperatures and shorter reaction times, a significant proportion of the mono-intermediate was obtained, indicating incomplete reaction. Based on void volume, the reaction times employed herein ranged from 1.2 to 4.8 min.

This is a significant decrease in reaction time and yield when compared to the analogous batch reaction, which achieved a ˜85% conversion in 30 minutes.

1. A process for preparing a compound of formula (I), 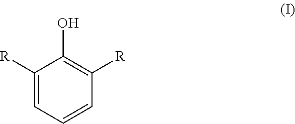
wherein R represents phenyl, substituted aryl, alkyl, or substituted alkyl,
comprising, reacting a compound of formula (II) 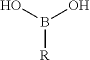
wherein R represents phenyl, substituted aryl, alkyl, or substituted alkyl,
with a compound of formula (III) 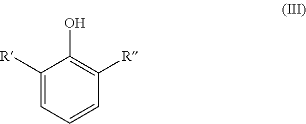
wherein R′ and/or R″ represents a halogen,
in the presence of a catalyst, which process is a double coupling of a boronic acid and a 2,6-dihalogenphenol in a Suzuki-Miyaura reaction on a sterically hindered position.

2. The process according to claim 1, wherein one or both of the halogens is iodine, bromide or chloride.

3. The process according to claim 1, wherein the compound of formula (I) is 2,6-diphenylphenol, the compound of formula (II) is phenyl boronic acid and the compound of formula (III) is 2,6-diiodophenol.

4. The process according claim 1, wherein the catalyst is a palladium catalyst.

5. The process according claim 1, wherein the catalyst is SiliaCat-DPP-Pd.

6. The process according claim 1, wherein the reaction takes place in a continuous flow system.

7. The process according claim 1, wherein the reaction time is less then 6 hours.

8. The process according claim 1, wherein the yield of the compound of formula (III) is at least 75%, 80%, 85%, 90%, 95%, 96%, 97%, 98%, 99%, 99.5%, 99.9%, or 100% by weight.

9. Composition A composition obtained by the process according to claim 1.

10. A method for preparing poly(2,6-diphenylphenylene oxide), the method comprising using a composition according to claim 9.

11. The method according to claim 10, wherein the method is a method for the manufacture of dyes, drugs, plastics, insulating materials, and/or insecticides.

12. The method according to claim 10, wherein the method is applied in medical applications or material research.

13. A method for preparing a 2,6-substituted phenol, in particular 2,6-diphenylphenol, the method comprising using a palladium catalyst.

14. The method according to claim 13, wherein the preparation of the 2,6-substituted phenol involves a double coupling of a boronic acid and a 2,6-dihalogenphenol in a Suzuki-Miyaura reaction on a sterically hindered position, in particular on the sterically hindered ortho positions of the 2,6-dihalogenphenol.

15. The method according to claim 13, wherein the palladium catalyst is SiliaCat-DPP-Pd.
